(19) United States
(12) Patent Application Publication
(10) Pub. No.: US 2024/0050557 A1
(43) Pub. Date: Feb. 15, 2024

(54) CORONAVIRUS VACCINE AND METHOD FOR PREPARATION THEREOF

(71) Applicant: BHARAT BIOTECH INTERNATIONAL LIMITED, Hyderabad (IN)

(72) Inventors: Deepak KUMAR, Hyderabad (IN); Krishna Murthy ELLA, Hyderabad (IN)

(73) Assignee: BHARAT BIOTECH INTERNATIONAL LIMITED, Hyderabad (IN)

(21) Appl. No.: 18/021,972

(22) PCT Filed: Aug. 21, 2021
(86) PCT No.: PCT/IN2021/050806

§ 371 Date: Feb. 17, 2023

(30) Foreign Application Priority Data
Aug. 21, 2020 (IN) 202041007559

(51) Int. Cl. A61K 39/215; A61K 39/39; A61P 37/04; C12N 7/00

(52) U.S. Cl. CPC A61K 39/215; A61K 39/39; A61P 37/04; C12N 7/00; A61K 2039/5252

(57) ABSTRACT

The present invention relates to vaccine and treatment of novel coronavirus (SARS-CoV-2) infection (COVID-19) in mammals. Particularly, the invention relates to coronavirus vaccine and method for preparation thereof. More particularly, the present invention discloses preparation of coronavirus vaccine comprising an inactivated, purified SARS-CoV2 as active ingredient. The present invention also discloses a method for preparation of killed-inactivated SARS-CoV-2 virus which is used as antigen in the vaccine composition. The present invention further relates to the method of antigen preparation including inactivation and purification of virus, SARS-CoV-2 vaccine preparation, composition, formulation and use of the same to elicit immune response against the SARS-CoV-2 in mammals, and it is also suitable for immunizing human subjects.

### RELATED PATENT APPLICATION

This application claims the priority to and benefit of Indian Patent Application No. 202041007559 filed on Aug. 21, 2020; the disclosures of which are incorporated herein by reference.

### FIELD OF THE INVENTION

The present invention relates to coronavirus vaccine and method for preparation thereof. Particularly present invention relates to vaccine and treatment of novel coronavirus (SARS-CoV-2) infection (COVID-19) in mammals. More particularly, the present invention relates to preparation of coronavirus vaccine comprising an inactivated, purified SARS-CoV2 as active ingredient. The present invention relates to a method for preparation of killed-inactivated purified SARS-CoV-2 virus bulk used as antigen for vaccine composition. The present invention also relates to the method of SARS-CoV-2 vaccine preparation and use of the same to elicit immune response against the SARS-CoV-2 in mammals, and it is also suitable for immunizing human subjects.

### BACKGROUND OF THE INVENTION

SARS-CoV-2

The novel corona virus (SARS-CoV-2) is an emerging pathogen, which belongs to the genus Betacoronavirus of family Coronaviridae. The genome is linear single-stranded positive sense RNA of approximately 29 Kilobases in size. Coronaviruses are enveloped, spherical in shape and are about 120 nm in diameter. As the genome is positive sense, it is infectious and acts as viral messenger RNA. Coronavirus viruses are found in a wide variety of animals and can cause respiratory and enteric disorders of diverse severity. The 30 genome analysis reveals that the SARS-CoV-2 is closely related to the bat coronavirus than the SARS-CoV or MERS-CoV.

Coronaviruses are associated with common cold in humans with typically mild to moderate symptoms also called as common cold. These infections usually subside without 35 causing serious health problems. However, the outbreaks of Severe Acute Respiratory Syndrome (SARS) and Middle East Respiratory Syndrome (MERS) have together resulted in more than 10,000 human infections with more than 1,500 fatalities. In distinct contrast to the mild human coronaviruses, infection with SARS-CoV frequently resulted in severe symptoms including fever, dry cough, shortness of breath and pneumonia.

Coronavirus Disease (COVID-19)

Recently, the emergence of a novel severe acute respiratory syndrome coronavirus (SARS-CoV-2) has led to the COVID-19 pandemic. The disease caused by the SARS-CoV-2 is named as COVID-19. The novel coronavirus (SARS-CoV-2) emerged from Wuhan, China in December 2019 and has spread rapidly among humans and is currently reported from 220 countries and territories around the world. Till date, more than 203,295,170 confirmed human infections were reported with more than 4,303,515 fatalities. These numbers are greater than that of SARS and MERS combined. In the COVID-19 outbreak, it was observed that the transmission of the SARS-CoV-2 happen before the appearance of the symptoms. This attributed to the increased number of human infections. The lack of protective immunity against coronaviruses makes the SARS-CoV-2 a potential threat.

Vaccines Against COVID-19

A number of SARS- and MERS-CoV vaccine candidates have been tested in animal models, including whole-inactivated, subunit, DNA, mRNA, viral-vectored, and live attenuated vaccines. Most of these vaccine candidates elicit neutralizing antibodies to the coronaviruses spike (S) protein, the major viral antigen. Neutralizing antibodies to the coronavirus spike protein are protective. However, T-cell mediated immune responses also appear to play a role in protection from lethal coronavirus challenges. An ideal vaccine candidate for coronavirus should elicit mucosal, humoral and cell mediated immune response to prevent coronavirus infection.

The SARS-CoV-2 is studded with spike protein which is utilized by the virus for its entry into the host cells. Multiple vaccine candidates are developed against COVID-19. Various platforms are being utilized for the development of the COVID-19 vaccine. The viral vectored, DNA and RNA vaccines target the spike protein and produce immune response against it. Inactivated vaccine on the other hand boosts immune response against the structural proteins of the SARS-CoV-2. However, there are very few inactivated vaccine candidates for COVID-19.

The current patent describes the preparation and formulation of vaccine candidates to prevent SARS-CoV-2 infections. Specifically, the current patent describes the production of inactivated purified SARS-CoV2 bulk which is ready to be formulated with a suitable adjuvant to prevent SARS-CoV-2 infections.

### OBJECTS OF THE INVENTION

Primary object of the invention is to provide a coronavirus vaccine and method for preparation thereof. It is the objective of the invention to provide a method for preparation of coronavirus vaccine comprising an inactivated, purified SARS-CoV2 as active ingredient.

Another object of the invention is to provide a method for preparation of inactivated purified SARS-CoV-2 virus bulk.

Another object of the invention is to provide methods for inactivation of SARS-CoV-2 by chemical means with formalin, beta-propiolactone and hydrogen peroxide and by physical means such as heat, gamma irradiation and ultraviolet light, preferably by chemical means such as beta-propiolactone inactivation.

Another object of the invention is to provide methods for purification of SARS-CoV-2.

Another object of the invention is to provide a method of production of inactivated purified SARS-CoV-2, wherein the inactivated purified SARS-CoV-2 obtained in the virus bulk is 25 a killed-inactivated purified SARS-CoV-2 bulk which is used as an active ingredient in the preparation of an immunogenic composition.

Another object of the invention is to provide stable immunogenic composition for prophylaxis of COVID-19 or any other coronavirus infections.

Another object of the invention is to provide a vaccine that can be used to elicit immune responses against SARS-CoV-2 in mammals.

It is the objective of the invention to provide methods for the formulation of killed-35 inactivated SARS-CoV-2 vaccine without adjuvants.

Yet another object of the invention is to provide a method to administer the antigen through intranasal, intraperitoneal, oral, intramuscular, subcutaneous and intradermal routes.

A further object of the invention is to provide a method of treatment and/or prophylaxis for COVID-19 and/or eliciting an immune response against SARS-CoV-2 and its variants in mammals including human subjects.

### SUMMARY OF THE INVENTION

The present invention relates to coronavirus vaccine and method for preparation thereof. The invention discloses the manufacturing process and stable vaccine composition.

In one aspect, the present invention provides a method for preparation of coronavirus vaccine.

A method for preparation of coronavirus vaccine comprising an inactivated, purified SARS-CoV2 as active ingredient, wherein inactivated purified SARS-CoV2 bulk used in the vaccine composition is prepared by a method comprising the following steps:
(a) using Vero cell line (with or without serum) as cell substrate for SARS-CoV-2 virus culture,
(b) scaling up the SARS-CoV-2 virus culture of step (a) upto a harvest volume of 10L and above,
(c) inactivating the SARS-CoV-2 virus culture of step (b), and
(d) purifying the SARS-CoV-2 virus culture obtained in step (c) and obtaining the inactivated purified SARS-CoV2 bulk.

In the said process the inactivated purified SARS-CoV-2 obtained is killed-inactivated purified SARS-CoV-2.

The Vero cell line used in step (a) of said process is African Green Monkey Kidney cells.

The optimum conditions of growth were determined for the propagation of SARS-CoV-2 in the mammalian cells.

The temperature condition of step (a) of said process ranges from 33° C. to 37° C. for the propagation of SARS-CoV2 in mammalian cells.

The multiplicity of infection of SARS-CoV2 in step (a) of said process ranges from 0.001 to 0.1 for the infection of the mammalian cells.

Further, a harvest time in step (a) of said process ranges between 48 hours to 80 hours post SARS-CoV2 infection of mammalian cells.

The inactivation method in step (c) of the said process is selected from a group consisting of formalin inactivation, beta propiolactone inactivation, heat inactivation, UV inactivation, gamma radiation inactivation, in the presence or absence of virus stabilizing agents.

The SARS-CoV-2 is inactivated by one of the following methods selected from:
i) Formalin treatment at any concentration ranging from 1: 500 upto 1: 4000 v/v of formalin: virus, at 8° C. to 37° C., preferably 25±3° C., for at least 1 to 7 days;
ii) Formalin treatment at any concentration ranging from 1: 500 upto 1: 4000 v/v of formalin: virus, at 2° C. to 8° C. for at least 10 to 30 days;
iii) Beta propiolactone at any concentration ranging from 1: 500 upto 1: 4000 v/v of BPL: virus, for at least 24 to 48 hrs at temperatures ranging from 8° C. to 30° C., preferably 25±3° C., for 48 hrs;
iv) Beta propiolactone at any concentration ranging from 1: 500 upto 1: 4000 v/v of BPL: virus, at 2° C. to 8° C. for at least 3-7 days;
v) A combination of BPL and formalin at any aforementioned conditions, preferably BPL inactivation at 1: 3000 (PBL: virus v/v) for 24 hrs followed by formalin inactivation at 1:3000 (formalin:virus, v/v) for 24 to 48 hrs at 15° C. to 30° C., preferably 25±3° C.

The SARS-CoV-2 is inactivated by beta-propiolactone ranging between 1:2000 to 1:4000 at 2-8° C. for 24-32 hours.

The SARS-CoV-2 is inactivated by beta-propiolactone at 1:2500 or 1:4000 ratio at 5+3° C. for 32 hours.

The purification method step (d) of the said process comprises the use of size exclusion or affinity chromatography resins.

The size-exclusion chromatography resin comprises Captocore 700 or any other size-exclusion resins for the purification of the SARS-CoV2.

The affinity chromatography resin comprises Cellufine sulfate or any other affinity resins for the purification of the SARS-CoV2.

The purification method also comprises the use of ultracentrifugation.

Another aspect of the present invention is to provide a method of production of inactivated purified SARS-CoV-2 bulk comprising inactivation followed by a combination of size-exclusion or affinity chromatography and tangential flow filtration (TFF).

The inactivated purified SARS-CoV-2 obtained in the virus bulk is a killed-inactivated purified SARS-CoV-2 which is used as an active ingredient in the preparation of an immunogenic composition.

In the said method, the immunogenic composition comprises said killed-inactivated purified SARS-CoV-2 as active ingredient, wherein the said composition is formulated as a coronavirus vaccine with or without use of one or more pharmaceutically acceptable excipient(s) as adjuvant and/or stabilizing agents.

The adjuvant is selected from the group consisting of a) aluminum salts comprising aluminum hydroxide, aluminum phosphate, aluminum sulphate phosphate; b) inulin; c) algammulin which is a combination of inulin and aluminium hydroxide; d) 25 monophosphoryl lipid A (MPL); e) resiquimod; f) muramyl dipeptide (MDP); g) N-glycolyl dipeptide (GMDP); h) 50 polylC; i) CpG oligonucleotide; j) aluminum hydroxide with MPL; k) any water in oil emulsion; 1) any oil in water emulsion that contains one or more of the following constituents: squalene or its analogues or any pharmaceutically acceptable oil, tween-80, sorbitan trioleate, alpha-tocopherol, cholecalciferol and 30 aqueous buffer, or any of the analogues and derivatives of the molecules thereof i) two or more combination of any of the 5 aforementioned adjuvants when formulated with SARS-CoV-2 virus antigens enhance the immune response against the virus.

The adjuvant when used in the vaccine is aluminum hydroxide present in a concentration 35 range of 0.1 mg to 1.5 mg of aluminum per vaccine dose, preferably 0.25 mg to 0.5 mg aluminum per vaccine dose.

The stabilizing agents when used are selected from the group consisting of sorbitol, L-glycine, mannitol, L-glutamic acid, human serum albumin and combination thereof.

The said immunogenic composition further comprises of preservatives.

The preservative is 2-phenoxyethanol at a concentration of 2.5 to 5 mg/mL.

The said immunogenic composition comprises killed-inactivated, purified SARS-CoV-2 in buffer comprising of 100 mM Phosphate buffered saline as antigen.

In the said immunogenic composition the antigen content and pH is stable at −70° C.

Another aspect of the invention is to provide a coronavirus vaccine obtained by the said method.

Another aspect of the invention is to provide a method for preparation of killed-inactivated purified SARS-CoV2 bulk comprising the following steps:
(a) using Vero cell line (with or without serum) as cell substrate for SARS-CoV-2 virus culture,
(b) scaling up the SARS-CoV-2 virus culture of step (a) upto a harvest volume of 10L and above,
(c) inactivating the SARS-CoV-2 virus culture of step (b), and
(d) purifying the SARS-CoV-2 virus culture obtained in step (c) and obtaining the 25 killed-inactivated purified SARS-CoV2 bulk.

Another aspect of the invention is to provide a method of inactivation of SARS-CoV-2 virus selected from:
i) Formalin treatment at any concentration ranging from 1:500 upto 1: 4000 v/v of 30 formalin: virus, at 8° C. to 37° C., preferably 25±3° C., for at least 1 to 7 days;
ii) Formalin treatment at any concentration ranging from 1:500 upto 1: 4000 v/v of formalin: virus, at 2° C. to 8° C. for at least 10 to 30 days;
iii) Beta propiolactone at any concentration ranging from 1:500 upto 1: 4000 v/v of BPL: virus, for at least 24 to 48 hrs at temperatures ranging from 8° C. to 30° C., preferably 25±3° C., for 48 hrs;
iv) Beta propiolactone at any concentration ranging from 1:500 upto 1: 4000 v/v of BPL: virus, at 2° C. to 8° C. for at least 3-7 days;
v) A combination of BPL and formalin at any aforementioned conditions, preferably BPL inactivation at 1: 3000 (PBL: virus v/v) for 24 hrs followed by formalin inactivation at 1:3000 (formalin:virus, v/v) for 24 to 48 hrs at 15° C. to 30° C., preferably 25±3° C.

Yet another aspect of the present invention is to provide a method of treatment and/or prophylaxis for COVID-19 and/or eliciting an immune response against SARS-CoV-2 and its variants in mammals, wherein the said method comprises administration of immunogenic composition comprising killed-inactivated purified SARS-CoV-2 bulk by intranasal, intraperitoneal, oral, intramuscular, subcutaneous or intradermal routes.

### BRIEF DESCRIPTION OF FIGURES

FIG. 1: Growth kinetics of SARS-CoV-2 in Vero cells at 33° C. (Example 1).

FIG. 2: Growth kinetics of SARS-CoV-2 in Vero cells at 35° C. (Example 1).

FIG. 3: Growth kinetics of SARS-CoV-2 in Vero cells at 37° C. (Example 1).

FIG. 4: Inactivation kinetics of SARS-CoV-2 (Example 2).

FIG. 5: Purification by ultracentrifugation (Example 2).

FIG. 6: Western blot analysis of samples from downstream processing (Example 2).

FIG. 7: Protein profile of virus bulk purified by size exclusion chromatography (Example 2).

FIG. 8: Protein profile of virus bulk purified by ultracentrifugation (Example 2).

FIG. 9: Protein profile of virus bulk purified by affinity chromatography (Example 2).

FIG. 10: Process workflow for manufacture of killed-inactivated purified SARS-CoV2 bulk (Example 3).

FIG. 11: Western blot of virus bulk with specific antibodies (Example 4).

FIG. 12: Total protein stability of the killed-inactivated, purified SARS-CoV-2 virus bulk (Example 5).

FIG. 13: pH stability of the killed-inactivated, purified SARS-CoV-2 virus bulk (Example 5).

FIG. 14: Microneutralization titers of mice immunized with killed-inactivated, purified SARS-CoV-2 virus bulk (Example 5).

### DETAILED DESCRIPTION OF THE INVENTION

Present invention discloses coronavirus vaccine and method for preparation thereof.

The present invention is directed to composition and method of manufacture of vaccine formulations for prophylaxis and treatment of SARS-CoV-2 infections as well as infections caused by other coronaviruses such as SARS-CoV and MERS-CoV. More particularly, the present invention discloses a method for preparation of coronavirus vaccine comprising an inactivated, purified SARS-CoV2 as active ingredient.

In one aspect, the invention is directed to a method for preparation of inactivated purified SARS-CoV-2 virus bulk, which is used in the vaccine composition, wherein inactivated purified SARS-CoV-2 obtained is killed-inactivated purified SARS-CoV-2. Immunogenic or vaccine composition using said vaccine antigen, its production and formulation, its immunogenicity in animals through various routes of administration are described.

In another aspect, the invention is directed to vaccine compositions for prophylaxis and treatment of SARS-CoV-2 infections, wherein the said composition comprises of killed-inactivated purified SARS-CoV-2 virus.

The present invention provides a stable vaccine composition comprising killed-inactivated purified SARS-CoV-2 virus as antigen, wherein the said antigens being formulated with or without an adjuvant in pharmaceutically acceptable buffer, wherein the vaccine 25 composition elicits protective immune response to SARS-CoV-2 in mammals when administered through various routes.

The SARS-CoV-2 antigen compositions is effective against any beta coronavirus strains that share anywhere between 50% to 100% identity at any other region of the genome 30 particularly in the amino acid levels in the receptor binding domain of the spike glycoprotein.

In one aspect of the present invention the immunogenicity of above said killed-inactivated, purified SARS-CoV-2 vaccine candidate is evaluated in animals through various routes of administration such as intranasal, intraperitoneal, oral, intramuscular, subcutaneous and 35 intradermal routes.

The invention describes generation of killed-inactivated purified SARS-CoV-2 virus as antigen for the vaccine composition.

Antigen:

The present invention discloses a method for preparation of killed-inactivated purified SARS-CoV-2 bulk which is used as antigen in vaccine composition. The said antigen/antigenic component is the active ingredient of the COVID-19 vaccine, which is an inactivated, purified SARS-CoV2.

The present invention describes the production of killed-inactivated purified SARS-CoV2 bulk which is ready to be formulated with a suitable adjuvant to prevent SARS-CoV-2 infections.

Preparation Method:

A method of production of killed-inactivated purified SARS-CoV-2 bulk involves inactivation followed by virus purification.

Inactivation Method:

In another embodiment of the invention, inactivation method is selected from a group of formalin inactivation, beta propiolactone inactivation, heat inactivation, UV inactivation, gamma radiation inactivation, in the presence or absence of virus stabilizing agents.

In a preferred embodiment of the invention, amino acids were selected individually or in combination, from a group L-Histidine, L-Glutamic acid, L-Glycine and L-Aspartic acid and L-Glutamine and human serum albumin.

In one preferred embodiment the SARS-CoV-2 is inactivated by one of the following methods selected from:
i) Formalin treatment at any concentration ranging from 1: 500 upto 1: 4000 v/v of formalin: virus, at 8° C. to 37° C., preferably 25±3° C., for at least 1 to 7 days;
ii) Formalin treatment at any concentration ranging from 1: 500 upto 1: 4000 v/v of formalin: virus, at 2° C. to 8° C. for at least 10 to 30 days;
iii) Beta propiolactone at any concentration ranging from 1: 500 upto 1: 4000 v/v of 35 BPL: virus, for at least 24 to 48 hrs at temperatures ranging from 8° C. to 30° C., preferably 25±3° C., for 48 hrs;
iv) Beta propiolactone at any concentration ranging from 1: 500 upto 1: 4000 v/v of BPL: virus, at 2° C. to 8° C. for at least 3-7 days;
v) A combination of BPL and formalin at any aforementioned conditions, preferably BPL inactivation at 1: 3000 (PBL: virus v/v) for 24 hrs followed by formalin inactivation at 1: 3000 (formalin:virus, v/v) for 24 to 48 hrs at 15° C. to 30° C., preferably 25±3° C.

Hydrogen peroxide at any concentration from 0.1 to 3%, preferably 0.1 to 1% at any temperature from 20-30° C. for 5 minutes to 120 minutes.

In one embodiment, the inactivation of the SARS-CoV-2 by irradiating agent comprises inactivation by gamma irradiation by exposure from 20 kGy (Kilo Gray) up to 35 kGy, preferably 25 kGy to 30 kGy from a ⁶⁰Co source.

In another embodiment, the inactivation of the SARS-CoV-2 by irradiating agent comprises inactivation by UV irradiation by exposure to 254 nm for 30-60 minutes.

In a further embodiment, the virus is inactivated by heat treatment at a temperature between 50° C. to 65° C. for 30 min up to 2 hrs.

The buffer used in the invention may be selected from the list comprising of phosphate buffer, citrate buffer, phosphate citrate buffer, borate buffer, tris(hydroxymethyl) aminomethane (Tris) containing buffer, succinate buffer, buffers containing glycine or histidine as one of the buffering agents, wherein phosphate buffer is sodium phosphate buffer at concentration of 5 mM up to 200 mM of phosphate ions of any pH between 6.5 to pH 9, and optionally containing sodium chloride at a concentration of 50 to 200 mM.

The buffer maintains the pH in a liquid composition above pH 6.5, preferably above pH 7.0 throughout the bioprocess from viral culture up to preparation of purified bulk with or without inactivation of the virus.

In one embodiment, the inactivation of SARS-CoV-2 virus is carried out in the presence of a stabilizing agent selected from lactose, sucrose, trehalose, maltose, mannose, iso-maltose, raffinose, stachyose, lactobiose, sorbitol, mannitol, lactobionic acid, dextran, L-glycine, L-histidine, L-glutamic acid, L-aspartic acid and human serum albumin or combinations thereof. However, in one preferred embodiment, the stabilizing agent may be selected from:
(a) 2% sorbitol and 1% L-glycine;
(b) 1% sorbitol and 0.5% L-glycine;
(c) 1% mannitol and 0.5% L-glycine;
(d) 1% mannitol and 0.5% L-glutamic acid; and
(e) 1% sorbitol and 0.5% L-glycine, 1% human serum albumin.

In an exemplary embodiment, the inactivation of SARS-CoV-2 virus comprises inactivation of any genotype/strain/variant virus particles.

Preferably in the present invention, the inactivating agent and conditions includes the use of beta-propiolactone between 1:2000 to 1:4000 at 2-8° C. for 24-32 hours.

Inactivation kinetics of SARS-CoV-2 were further analyzed (Example 2.1).

Purification Method:

In another preferred embodiment of this invention, the purification method is selected by use of cellufine sulphate, DEAE-Sephadex, CM-Sephadex with salt gradient, by gel filtration on Captocore-700, Sepharose CL-4B, ceramic hydroxyapatite column with gradient of 0.2M to 0.8M phosphate followed by diafiltration, and ultracentrifugation on a 20-60% sucrose gradient, most preferably by Capto core 700 column.

In the preferred embodiment, the purification method includes the use of size exclusion or affinity chromatography resins. The size-exclusion chromatography resins such as Capto core 700 or any other size-exclusion resins is used for the purification of the SARS-CoV2. The affinity chromatography resins such as Cellufine sulfate or any other affinity resins is used for the purification of the SARS-CoV2.

The purification method also includes the use of ultracentrifugation.

Elutes or flowthroughs with the antigen were then concentrated through tangential flow filtration (TFF) column and diluted with phosphate buffer to obtain the drug substance.

In one preferred embodiment, a method of production of inactivated purified SARS-CoV-2 bulk comprises inactivation followed by a combination of size-exclusion or affinity chromatography and tangential flow filtration (TFF).

Method for Preparation of Coronavirus Vaccine:

The invention discloses a method for preparation of coronavirus vaccine comprising an inactivated, purified SARS-CoV2 as active ingredient.

The present invention is directed to a method for preparation of coronavirus vaccine comprising an inactivated, purified SARS-CoV2 as active ingredient, wherein inactivated purified SARS-CoV2 bulk used in the vaccine composition is prepared by a method comprising the following steps:
(a) using Vero cell line (with or without serum) as cell substrate for SARS-CoV-2 virus culture,
(b) scaling up the SARS-CoV-2 virus culture of step (a) upto a harvest volume of 10L and above,
(c) inactivating the SARS-CoV-2 virus culture of step (b), and
(d) purifying the SARS-CoV-2 virus culture obtained in step (c) and obtaining the inactivated purified SARS-CoV2 bulk.

The inactivated purified SARS-CoV-2 obtained though the above method is killed-inactivated purified SARS-CoV-2.

1) Vero Cell Line as Cell Substrate:

In the present invention, Vero cells (African Green Monkey Kidney cells) are used as cell substrate for SARS-CoV-2 virus culture. The Vero or Vero E6 or Vero-TMPRSS2 cells are propagated and infected with SARS-CoV-2.

Growth kinetics of SARS-CoV-2 in Vero cells were further analyzed (Example 1).

In the upstream process, optimum conditions of growth were determined for the propagation of SARS-CoV-2 in the mammalian cells.

The optimum upstream conditions include a temperature condition of 33° C. to 37° C. for the propagation of SARS-CoV2 in mammalian cells.

The optimum upstream conditions further include use of multiplicity of infection of SARS-CoV2 from 0.001 to 0.1 for the infection of the mammalian cells.

The optimum upstream conditions also include a harvest time between 48 hours to 80 hours post SARS-CoV2 infection of mammalian cells.

2) Scaling Up Procedures:

The scaling up procedures includes propagation of Vero cells by incubation, followed by seeding in the bioreactors.

In the downstream process of COVID-19 vaccine production, appropriate inactivating agent and conditions, and the purification methodologies were determined for the COVID-19 vaccine production.

3) Inactivation Method:

In another embodiment of the invention, inactivation method is selected from a group of formalin inactivation, beta propiolactone inactivation, heat inactivation, UV inactivation, gamma radiation inactivation, in the presence or absence of virus stabilizing agents.

In a preferred embodiment of the invention, amino acids were selected individually or in combination, from a group L-Histidine, L-Glutamic acid, L-Glycine and L-Aspartic acid and L-Glutamine and human serum albumin.

In one preferred embodiment the SARS-CoV-2 is inactivated by one of the following methods selected from:
i) Formalin treatment at any concentration ranging from 1: 500 upto 1: 4000 v/v of formalin: virus, at 8° C. to 37° C., preferably 25±3° C., for at least 1 to 7 days;
ii) Formalin treatment at any concentration ranging from 1: 500 upto 1: 4000 v/v of formalin: virus, at 2° C. to 8° C. for at least 10 to 30 days;
iii) Beta propiolactone at any concentration ranging from 1: 500 upto 1: 4000 v/v of BPL: virus, for at least 24 to 48 hrs at temperatures ranging from 8° C. to 30° C., preferably 25±3° C., for 48 hrs;
iv) Beta propiolactone at any concentration ranging from 1: 500 upto 1: 4000 v/v of BPL: virus, at 2° C. to 8° C. for at least 3-7 days;
v) A combination of BPL and formalin at any aforementioned conditions, preferably BPL inactivation at 1: 3000 (PBL: virus v/v) for 24 hrs followed by formalin inactivation at 1: 3000 (formalin:virus, v/v) for 24 to 48 hrs at 15° C. to 30° C., preferably 25±3° C.;

Hydrogen peroxide at any concentration from 0.1 to 3%, preferably 0.1 to 1% at any temperature from 20-30° C. for 5 minutes to 120 minutes.

In one embodiment, the inactivation of the SARS-CoV-2 by irradiating agent comprises inactivation by gamma irradiation by exposure from 20 kGy (Kilo Gray) up to 35 kGy, preferably 25 kGy to 30 kGy from a ⁶⁰Co source.

In another embodiment, the inactivation of the SARS-CoV-2 by irradiating agent comprises inactivation by UV irradiation by exposure to 254 nm for 30-60 minutes.

In a further embodiment, the virus is inactivated by heat treatment at a temperature between 50° C. to 65° C. for 30 min up to 2 hrs.

The buffer used in the invention may be selected from the list comprising of phosphate buffer, citrate buffer, phosphate citrate buffer, borate buffer, tris(hydroxymethyl) aminomethane (Tris) containing buffer, succinate buffer, buffers containing glycine or histidine as one of the buffering agents, wherein phosphate buffer is sodium phosphate buffer at concentration of 5 mM up to 200 mM of phosphate ions of any pH between 6.5 to pH 9, and optionally containing sodium chloride at a concentration of 50 to 200 mM.

The buffer maintains the pH in a liquid composition above pH 6.5, preferably above pH 7.0 throughout the bioprocess from viral culture up to preparation of purified bulk with or without inactivation of the virus.

In one embodiment, the inactivation of SARS-CoV-2 virus is carried out in the presence of a stabilizing agent selected from lactose, sucrose, trehalose, maltose, mannose, iso-30 maltose, raffinose, stachyose, lactobiose, sorbitol, mannitol, lactobionic acid, dextran, L-glycine, L-histidine, L-glutamic acid, L-aspartic acid and human serum albumin or combinations thereof. However, in one preferred embodiment, the stabilizing agent may be selected from:
(a) 2% sorbitol and 1% L-glycine;
(b) 1% sorbitol and 0.5% L-glycine;
(c) 1% mannitol and 0.5% L-glycine;
(d) 1% mannitol and 0.5% L-glutamic acid; and
(e) 1% sorbitol and 0.5% L-glycine, 1% human serum albumin.

In an exemplary embodiment, the inactivation of SARS-CoV-2 virus comprises inactivation of any genotype/strain/variant virus particles.

Preferably in the present invention, the inactivating agent and conditions includes the use of beta-propiolactone between 1:2000 to 1:4000 at 2-8° C. for 24-32 hours.

Inactivation kinetics of SARS-CoV-2 were further analyzed (Example 2.1).

4) Purification Method:

In another preferred embodiment of this invention, the purification method is selected by use of cellufine sulphate, DEAE-Sephadex, CM-Sephadex with salt gradient, by gel filtration on Captocore-700, Sepharose CL-4B, ceramic hydroxyapatite column with gradient of 0.2M to 0.8M phosphate followed by diafiltration, and ultracentrifugation on a 20-60% sucrose gradient, most preferably by Capto core 700 column.

In the preferred embodiment, the purification method includes the use of size exclusion or affinity chromatography resins. The size-exclusion chromatography resins such as Capto core 700 or any other size-exclusion resins is used for the purification of the SARS-CoV2. The affinity chromatography resins such as Cellufine sulfate or any other affinity resins is used for the purification of the SARS-CoV2.

The purification method also includes the use of ultracentrifugation.

Elutes or flowthroughs with the antigen were then concentrated through TFF column and diluted with phosphate buffer to obtain the drug substance.

In one preferred embodiment, the present invention provides a method for preparation of killed-inactivated purified SARS-CoV2 bulk comprising the following steps:
(a) using Vero cell line (with or without serum) as cell substrate for SARS-CoV-2 virus culture,
(b) scaling up the SARS-CoV-2 virus culture of step (a) upto a harvest volume of 10L and above,
(c) inactivating the SARS-CoV-2 virus culture of step (b), and
(d) purifying the SARS-CoV-2 virus culture obtained in step (c) and obtaining the killed-inactivated purified SARS-CoV2 bulk.

Vaccine Composition:

The killed-inactivated purified SARS-CoV-2 bulk produced through the above method is used as an active ingredient in the preparation of an immunogenic composition or vaccine composition and the said composition can be used to prevent disease and/or infection with SARS-CoV-2.

In one embodiment the said immunogenic composition comprises antigen as an active ingredient along with pharmaceutically acceptable excipients.

The thus produced vaccine candidate is further provided as an immunogenic composition or vaccine composition comprising an immunogenically effective concentration of vaccine candidate sufficient to elicit desires result with or without one or more pharmaceutically acceptable excipients(s).

The vaccine composition of the invention is obtained by a process wherein neutralizing antibodies are largely elicited against the spike glycoprotein such as in optimally inactivated virus.

Antigen:

The present invention discloses the stable vaccine composition of killed-inactivated purified SARS-CoV-2 virus as antigen.

The said antigen is obtained by the method which involves inactivation followed by a combination of size-exclusion or affinity chromatography and TFF.

Antigen Concentration:

The total protein in the purified bulk ranges from 150 to 200 microgram/mL. The SARS-CoV-2 specific protein Spike S1 in the bulk ranges from 5-50 microgram/mL.

Excipient(s):

The composition comprising killed-inactivated purified SARS-CoV-2 of the present invention generally may comprise and/or formulated with or without one or more pharmaceutically acceptable excipient(s), suitable for vaccine composition or formulation to be administered in mammals through various routes of administration in suitable concentration.

The vaccine composition of the invention may further comprise an adjuvant, wherein the adjuvant is selected from the group consisting of a) aluminum salts comprising aluminum hydroxide, aluminum phosphate, aluminum sulphate phosphate; b) inulin; c) algammulin which is a combination of inulin and aluminium hydroxide; d) monophosphoryl lipid A (MPL); e) resiquimod; f) muramyl dipeptide (MDP); g) N-glycolyl dipeptide (GMDP); h) 50 polyIC; i) CpG oligonucleotide; j) aluminum hydroxide with MPL; k) any water in oil emulsion; 1) any oil in water emulsion that contains one or more of the following constituents: squalene or its analogues or any pharmaceutically acceptable oil, tween-80, sorbitan trioleate, alpha-tocopherol, cholecalciferol and aqueous buffer, or any of the analogues and derivatives of the molecules thereof i) two or more combination of any of the 5 aforementioned adjuvants when formulated with SARS-CoV-2 virus antigens enhance the immune response against the virus.

In one preferred embodiment the composition comprises aluminum hydroxide in a concentration range of 0.1 mg to 1.5 mg of aluminum per vaccine dose, preferably 0.25 mg to 0.5 mg aluminum per vaccine dose.

The adjuvant of the composition of the invention confers mucosal immunity and systemic immunity when administered in mammals.

In another embodiment of the invention, the formulations are prepared with excipients and preservatives.

In another embodiment of the invention, stabilizing agents in the vaccine formulation were used individually or in combination of sorbitol, L-glycine, mannitol, L-glutamic acid and human serum albumin in various concentrations was used to study the same.

The vaccine composition of the invention optionally comprises 2-phenoxyethanol as preservative at a concentration of 2.5 to 5 mg/mL.

The said immunogenic composition comprises Antigen: active ingredient inactivated, purified SARS-CoV-2 in buffer comprising of 100 mM Phosphate buffered saline.

In another embodiment of the invention, the potency of the vaccine formulations have been tested in animal models to show complete protection from viremia over a whole range of dosage.

In another embodiment of the invention, the candidate vaccine can be administered either as a single dose or in two or more doses by intranasal, intraperitoneal, oral, intramuscular, subcutaneous or intradermal routes in animals and humans to elicit the immune response.

In another embodiment of the invention, the candidate vaccine can be administered followed by vaccination with another vaccine by intranasal, intraperitoneal, oral, intramuscular, subcutaneous or intradermal routes in animals and humans to elicit the immune response.

In another embodiment of the invention, assays for neutralizing antibody titers were conducted to check the neutralizing antibody levels against vaccine formulations of the present invention which has shown to elicit the high level of neutralizing antibodies.

The vaccine composition with SARS-CoV-2 virus antigen is administered at any dose ranging from 0.125 pg to 100 μg per dose with or without an adjuvant, either as a single dose or in two or more doses to elicit an immune response in mammals.

Single or multiple doses of the inactivated vaccine composition elicits predominantly antibody and cell-mediated immune response against SARS-CoV-2 virus and is suitable for administration to humans.

In another embodiment of the invention, the vaccine composition is present in the liquid form.

The present invention provides a stable vaccine composition comprising killed-inactivated purified SARS-CoV-2 as an active ingredient, wherein the said antigens being formulated with or without an adjuvant in pharmaceutically acceptable buffer, wherein the vaccine composition elicits protective immune response to SARS-CoV-2 in mammals, when administered through various routes. The SARS-CoV-2 antigen composition is effective 35 against any beta coronavirus strains that share anywhere between 50% to 100% identity at any other region of the genome particularly in the amino acid levels in the receptor binding domain of the spike glycoprotein.

In the stable immunogenic composition the antigen content and pH is stable at −70° C.

Administration of the Vaccine Candidate:

The killed-inactivated purified SARS-CoV-2 vaccine candidate can be administered to animals and humans through intranasal, intraperitoneal, oral, intramuscular, subcutaneous or intradermal routes to test the immunogenicity.

In one embodiment of the invention, the candidate vaccine can be administered either as a single dose or in two or more doses by intranasal, intraperitoneal, oral, intramuscular, subcutaneous or intradermal routes in animals and humans to elicit the immune response.

In another embodiment of the invention, the candidate vaccine can be administered followed by vaccination with another vaccine by intranasal, intraperitoneal, oral, intramuscular, subcutaneous or intradermal routes in animals and humans to elicit the immune response.

In another embodiment of the invention, assays for neutralizing antibody titers were conducted to check the neutralizing antibody levels against vaccine formulations of the present invention which has shown to elicit the high level of neutralizing antibodies.

Method of Treatment:

In another aspect the invention discloses a method of treatment and/or prophylaxis for COVID-19 and/or eliciting an immune response against SARS-CoV-2 and its variants in mammals, wherein the said method comprises administration of immunogenic composition comprising killed-inactivated purified SARS-CoV-2 bulk by various routes.

In one embodiment the invention provides a method of eliciting a protective immune response in mammals including humans comprising administering the vaccine composition of by any route comprising intramuscular, intradermal, subcutaneous, intravenous, oral, intranasal or transcutaneous routes.

Single or multiple doses of the killed-inactivated purified SARS-CoV-2 vaccine composition elicits predominantly antibody and cell-mediated immune response against SARS-CoV-2 virus and is suitable for administration to humans.

The composition of the invention may be administered by any method comprising needles and syringes including pre-filled syringes, microneedle patch, needle-free patch, inhalation and nasal sprays.

The invention also provides a method of in vitro or in vivo use of the SARS-CoV-2 virus antibodies of the composition for preparation of immunodiagnostic and immunotherapeutic reagents for SARS-CoV-2 virus infections.

The vaccine composition, immunogenic composition and method of treatment as described above, wherein the vaccine composition with SARS-CoV-2 virus antigen is administered at any dose ranging from 0.125 pg to 100 μg per dose with or without an adjuvant, either as a single dose or in two or more doses to elicit an immune response in mammals.

In one of the preferred embodiments, present invention provides a method of treatment and/or prophylaxis for COVID-19 and/or eliciting an immune response against SARS-CoV-2 and its variants in mammals including human subjects, wherein the said method comprises intramuscular administration of a vaccine formulation in dose range of 1 to 10 microgram of antigen per dose.

In another preferred embodiment, the drug substance was diluted 10 times and 0.5 mL of the diluted drug substance and was injected through intraperitoneal or subcutaneous route.

### EXAMPLES

The above-described aspects of the invention further be understood by following non-limiting examples and corresponding drawing figures.

### Example-1: Virus: Propagation, Growth and Culture Preparation

Propagation of Virus:

Passage stability of the virus SARS-CoV-2 strain (NIV-2020-770) was determined by subjecting three drug substance batches and passages (working virus bank, virus inoculum, 10, 12, 14, 17) of the SARS-CoV-2 virus in Vero cells to Next-Generation sequencing and the sequences were aligned with the reference sequences [3rd passage of SARS-CoV-2 strain (NIV-2020-770), Severe acute respiratory syndrome coronavirus 2 isolate SARS-CoV-2/human/ITA/APU-POLBA01/2020 (MW450666.1) and hCoV-19/South Africa/KRISP-K004627/2020 (Accession ID: EPI_ISL_660643)].

TABLE A
Mapping and consensus statistics with 3^{rd} passage of SARS-
CoV-2 strain (NIV-2020-770) as the reference sequence.

| | | | Genome | | Total length |
|---|---|---|---|---|---|
| Sample | Reference | Mapping | Coverage | No. of | of consensus |
| Name | Organism | (%) | (%) | Sequence(s) | (bp) |
| 37620001A | nCov-770- | 19.27 | 100 | 1 | 29,885 |
| 37620002A | Passage 3 | 40.46 | 100 | 1 | 29,885 |
| 37620003A | | 52.60 | 100 | 1 | 29,885 |
| Paggage_10 | | 29.74 | 100 | 1 | 29,885 |
| Paggage_12 | | 17.08 | 100 | 1 | 29,885 |
| Paggasge_14 | | 78.82 | 100 | 1 | 29,885 |
| Passage_17 | | 98.71 | 100 | 1 | 29,885 |
| Working_Virus | | 86.82 | 99.99% | 1 | 29,885 |
| Virus Inoculum | | 57.96 | 100% | 1 | 29,885 |

TABLE B
Mapping and consensus statistics with severe acute respiratory syndrome
coronavirus 2 isolate SARS-CoV-2/human/ITA/APU-POLBA01/2020 (MW450666.1)

| | | | Genome | | Total length |
|---|---|---|---|---|---|
| Sample | Reference | Mapping | Coverage | No. of | of consensus |
| Name | Organism | (%) | (%) | Sequence(s) | (bp) |
| 37620001A | Severe acute | 19.26 | 100 | 1 | 29,780 |
| 37620002A | respiratory | 40.45 | 100 | 1 | 29,780 |
| 37620003A | syndrome | 52.58 | 100 | 1 | 29,780 |
| Paggage_10 | coronavirus 2 | 29.72 | 100 | 1 | 29,780 |
| Paggage_12 | isolate SARS- | 17.06 | 100 | 1 | 29,780 |
| Paggasge_14 | CoV-2/human/ITA/ | 78.81 | 100 | 1 | 29,780 |
| Passage_17 | APU-POLBA01/2020 | 98.71 | 100 | 1 | 29,780 |
| Working_Virus | (MW450666.1) | 86.82 | 100 | 1 | 29,780 |
| Virus Inoculum | | 57.95 | 100 | 1 | 29,780 |

TABLE C
Mapping and consensus statistics with hCoV-19/South Africa/KRISP-
K004627/2020 (Accession ID: EPI_ISL_660643)

| | | | Genome | | Total length |
|---|---|---|---|---|---|
| Sample | Reference | Mapping | Coverage | No. of | of consensus |
| Name | Organism | (%) | (%) | Sequence(s) | (bp) |
| 37620001A | hCoV-19/South | 19.36 | 100 | 1 | 29,901 |
| 37620002A | Africa/KRISP- | 40.51 | 100 | 1 | 29,901 |
| 37620003A | K004627/2020 | 52.66 | 100 | 1 | 29,901 |
| Paggage_10 | (Accession ID: | 29.82 | 100 | 1 | 29,901 |
| Paggage_12 | EPI_ISL_660643) | 17.16 | 100 | 1 | 29,901 |
| Paggasge_14 | | 78.84 | 100 | 1 | 29,901 |
| Passage_17 | | 98.72 | 100 | 1 | 29,901 |
| Working_Virus | | 86.84 | 100 | 1 | 29,901 |
| Virus Inoculum | | 58.00 | 100 | 1 | 29,901 |

Scale Up Method:

The Vero cells were propagated by incubating at temperatures between 33° C. to 37° C. The Vero cells were revived in 1× Cell stack (CS) 1 which were expanded to 1×CS 10 after 3-4 days of incubation. The Vero cells from 1×CS 10 were expanded to 10×CS 10 and incubated for 3-4 days. Twenty CS 40 are seeded with Vero cells from 10×CS 10, the cells are incubated for 3-4 days. Finally, the bioreactors with cytodex beads or other cell adherent matrix are seeded with the Vero cells from 20×CS 40 and incubated to propagate it. The Vero cells were infected once 80-90% of cell density was achieved on the matrix.

### Example-1.1: Growth Kinetics of SARS-CoV-2 in Vero Cells

Growth Kinetics Characterization:

1.1.1: Growth Kinetics of SARS-CoV-2 in Vero Cells at 33° C.

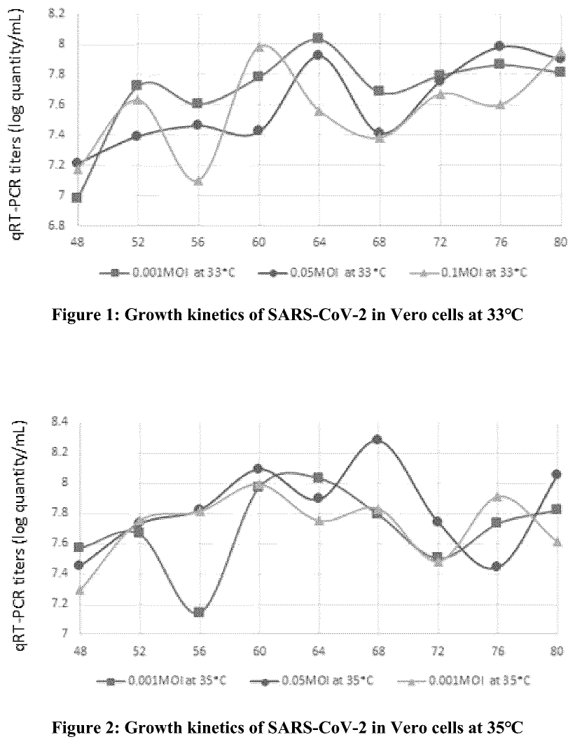

Vero cells at the rate of 10⁶ cells were seeded per 25 cm² tissue culture flasks and incubated overnight at 37° C. The cells were then infected with three different multiplicity of infection (0.001, 0.05, 0.1) of SARS-CoV-2 virus and again incubated at 33° C. Harvests at every 4 hours (h) from 48 h to 80 h were tested by quantitative RT-PCR specific for RNA-dependent RNA polymerase (RdRp) gene of SARS-CoV-2 virus. As a standard for quantitative RT-PCR, synthetic RdRp gene with known copy number was used. The results were plotted as line graph representing the virus titer at each time point. The result is depicted and shown in FIG. 1.

1.1.2: Growth Kinetics of SARS-CoV-2 in Vero Cells at 35° C.

Vero cells at the rate of 10⁶ cells were seeded per 25 cm² tissue culture flasks and incubated overnight at 37° C. The cells were then infected with three different multiplicity of infection (0.001, 0.05, 0.1) of SARS-CoV-2 virus and again incubated at 35° C. Harvests at every 4 hours (h) from 48 h to 80 h were tested by quantitative RT-PCR specific for RNA-dependent RNA polymerase (RdRp) gene of SARS-CoV-2 virus. As a standard for quantitative RT-PCR, synthetic RdRp gene with known copy number was used. The results were plotted as line graph representing the virus titer at each time point. The result is depicted and shown in FIG. 2.

1.1.3: Growth Kinetics of SARS-CoV-2 in Vero Cells at 37° C.

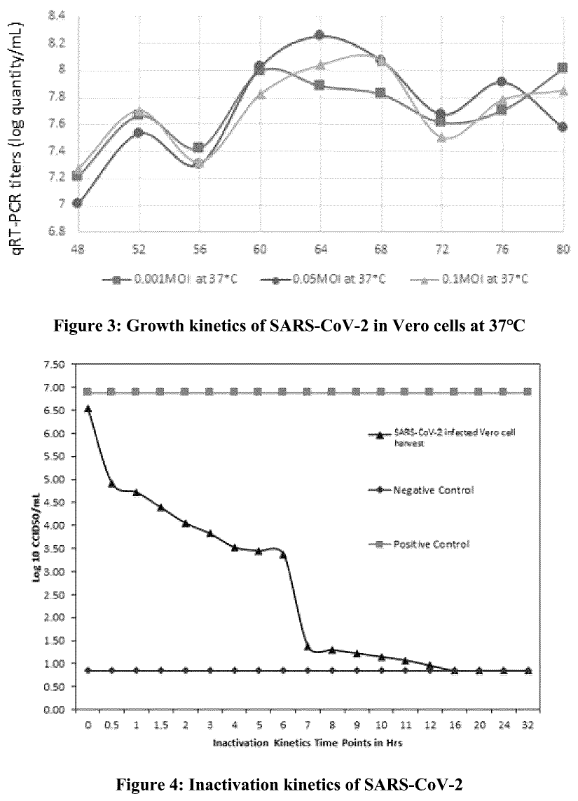

Vero cells at the rate of 10⁶ cells were seeded per 25 cm² tissue culture flasks and incubated overnight at 37° C. The cells were then infected with three different multiplicity of infection (0.001, 0.05, 0.1) of SARS-CoV-2 virus and again incubated at 37° C. Harvests at every 4 hours (h) from 48 h to 80 h were tested by quantitative RT-PCR specific for RNA-dependent RNA polymerase (RdRp) gene of SARS-CoV-2 virus. As a standard for quantitative RT-PCR, synthetic RdRp gene with known copy number was used. The results were plotted as line graph representing the virus titer at each time point. The result is depicted and shown in FIG. 3.

### Example-2: Antigen Production Method

A method of production of killed-inactivated, purified SARS-CoV-2 bulk involves inactivation followed by virus purification.

### Example-2.1: Inactivation of Virus

Inactivation methods involves formalin inactivation, beta propiolactone inactivation, heat inactivation, UV inactivation, gamma radiation inactivation, etc. Present method for inactivation of SARS-CoV-2 involves chemical means such as beta-propiolactone.

Inactivation Using Beta-Propiolactone:

Viral harvest is clarified with 0.8-micron and 0.45-micron filters. Five percent sucrose and beta-propiolactone at 1:2500 or 1:4000 ratio were added to the clarified harvest. Virus inactivation is carried out in a vessel at 5+3° C. for 16 hours with continuous stirring. The contents of the vessel are transferred to another vessel and the inactivation is continued at 5+3° C. for 16 hours with continuous stirring. Post inactivation, the beta-propiolactone is hydrolysed at 36+1° C. for 2 hours.

Inactivation Kinetics of SARS-CoV-2:

The SARS-CoV-2 harvest was inactivated with β-propiolactone (1:2500) by stirring at 2-8° C. for 16 hours. Infectious SARS-CoV-2 virus were estimated by CCID₅₀ at each time point from 0 hours to 32 hours. After 16 hours of inactivation, the sample is transferred to another vessel and kept for stirring at 2-8° C. for another 16 hours. The infectious titers of SARS-CoV-2 virus (Log 10 titers expressed in CCID₅₀/mL) on Y-axis are plotted against the time points on X-axis. The result is depicted and shown in FIG. 4.

### Example-2.2: Purification of Virus

Purification method involves Purification by size exclusion chromatography, Purification by ultracentrifugation and Purification by affinity chromatography resins such as Cellufine sulphate.

Purification by size exclusion chromatography: The column was washed with 2 to 5 column volumes of sterile water. About 2 to 4 column volumes of 30 mM to 60 mM phosphate buffer was used for washing or equilibration. Inactivated SARS-CoV-2 harvest was loaded on the column and the flow-through from the column was collected and concentrated and buffer exchanged with TFF.

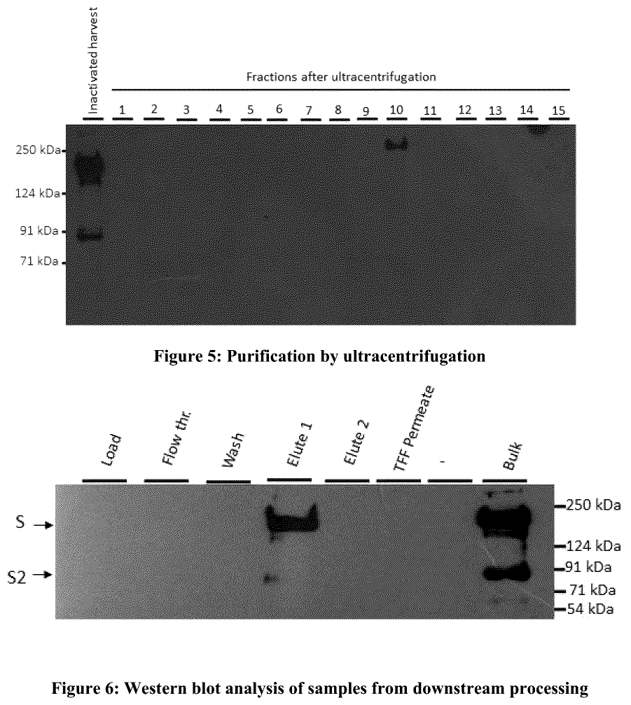

Purification by ultracentrifugation: The inactivated harvest was purified by ultracentrifugation. Sucrose gradient from 20% to 50% was prepared, samples were overlaid on the sucrose gradient and centrifuged at a speed of 100,000 to 120,000 g. The fractions from the bottom of the tube were collected and western blot was carried out with the Spike S2-specific rabbit polyclonal antibodies. The result is depicted and shown in FIG. 5.

Purification by affinity chromatography resins such as Cellufine sulphate: The column was washed with 3 to 6 column volumes of sterile water. About 2 to 4 column volumes of neutralization buffer consisting of 2 to 3 Molar of sodium chloride. The column was equilibrated with 4-6 column volumes of equilibration buffer. Ten to twenty column volumes of inactivated sample were loaded and the column was washed with equilibration buffer until the optical density at 280 nm of the washes reaches base line. Elution buffer is used to elute the SARS-CoV-2 bound to the affinity resin and this process is continued until the optical density at 280 nm reaches the base line. The column is neutralized with approximately 2-4 column volumes of sterile water and later sanitized with 0.5 M sodium hydroxide.

2.2.1: Western Blot Analysis of Samples from Downstream Processing:

The inactivated SARS-CoV2 harvests were subjected to purification with affinity chromatography resins such as cellufine sulphate. The samples during the affinity chromatography such as load, flow through, column wash, elute 1, elute 2 and TFF permeate, and the final bulk were resolved on 10% SDS-Polyacrylamide gel. The resolved proteins were transferred on PVDF membrane and western blot analysis was carried out with rabbit polyclonal spike S2-specific antibody. The bound rabbit antibody was detected using anti-rabbit IgG-peroxidase and the signal was developed by enhanced chemiluminescence reagent and the signals were captured on X-ray films. The results indicate the presence of SARS-CoV-2 specific full-length Spike (S) and Spike S2 fragment (S2) proteins in the Elute 1 and Bulk sample. The result is depicted and shown in FIG. 6.

2.2.2: Protein Profile of Virus Bulk Purified by Size Exclusion Chromatography:

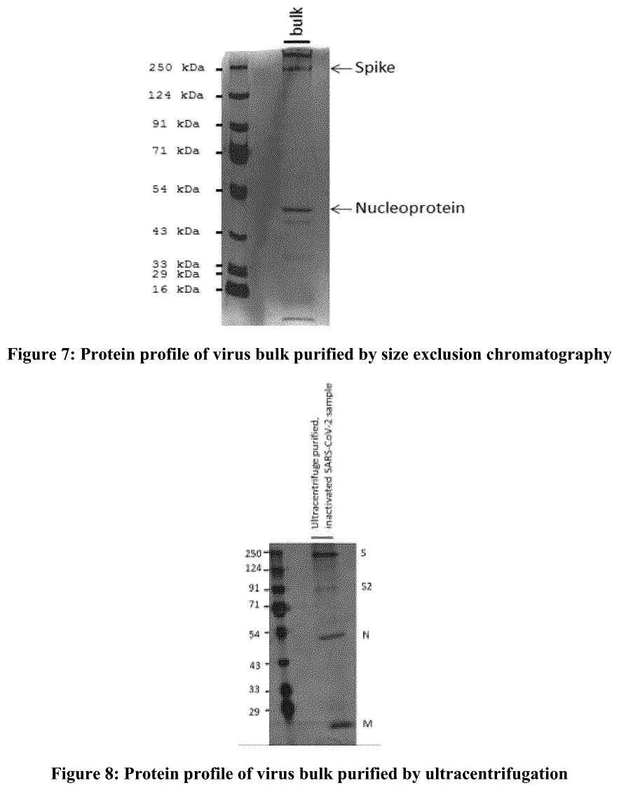

The bulk (killed-inactivated purified SARS-CoV-2 bulk) prepared by size exclusion chromatography such as Captocore-700 resins was loaded and resolved on 10% SDS-Polyacrylamide gels. The resolved proteins were visualized by silver staining. The result is depicted and shown in FIG. 7.

2.2.3: Protein Profile of Virus Bulk Purified by Ultracentrifugation:

The bulk (killed-inactivated SARS-CoV-2 bulk) prepared by ultracentrifugation of the inactivated SARS-CoV-2 on 20%-50% sucrose gradient. The bulk sample was resolved on 10% SDS-Polyacrylamide gels and the proteins in the resolved gels were visualized by silver staining. The result is depicted and shown in FIG. 8.

2.2.4: Protein Profile of Virus Bulk Purified by Affinity Chromatography:

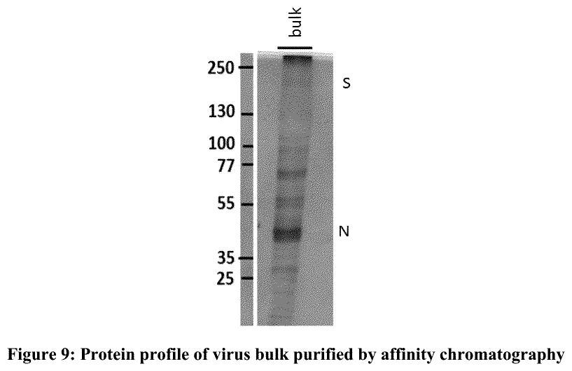

The bulk (killed-inactivated purified SARS-CoV-2 bulk) was resolved on 10% SDS-Polyacrylamide gels and the proteins in the resolved gels were visualized by silver staining. The result is depicted and shown in FIG. 9.

### Example-3: Killed-Inactivated Purified Sars-Cov2 Bulk Production Method

### Example-3.1: Process Workflow for Manufacture of Killed-Inactivated Purified SARS-CoV2 Bulk

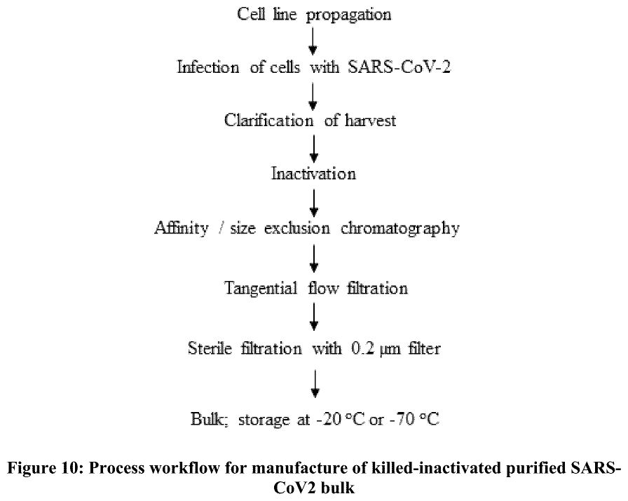

The manufacturing process for the production of the killed-inactivated SARS-CoV2 bulk is mentioned in the flow diagram FIG. 10.

The Vero or Vero E6 or Vero-TIVIPRSS2 cells were propagated in either bioreactors or cell stacks (CS) for manufacture of inactivated SARS-CoV2 vaccine. The Vero cells were propagated by incubating at temperatures between 33° C. to 37° C. The Vero cells were revived in 1×CS1 which were expanded to 1×CS10 after 3-4 days of incubation. The Vero cells from 1×CS10 were expanded to 10×CS10 and incubated for 3-4 days. Twenty CS40 are seeded with Vero cells from 10×CS10, the cells are incubated for 3-4 days. The cells propagated in 20×CS40 were either infected at this stage with SARS-CoV2 or the cells were expanded further to seed the bioreactors with cell adherent matrix such as cytodex beads. The cells were allowed to propagate and were infected with SARS-CoV2 once 80-90% of cell density was achieved on the cell adherent matrix. The SARS-CoV2 infected cells were harvested and clarified with 0.8μ followed by 0.2μ filters. The clarified harvest was inactivated with β-propiolactone (1:2500) by stirring at 2-8° C. for 16 hours. After 16 hours of inactivation, the clarified harvest is transferred to another vessel and kept for stirring at 2-8° C. for another 16 hours. The inactivated harvest was purified either by size exclusion chromatography or affinity chromatography. In size exclusion chromatography, the flow through is collected and further concentrated by Tangential Flow Filtration (TFF). The affinity chromatography elutes were further concentrated by TFF. The concentrated antigens were diluted with phosphate buffer and sterile filtered with 0.2 II. filters to obtain the killed-inactivated, purified SARS-CoV2 virus bulk. The bulk are stored at −20° C. or −70° C.

### Example-4: Western Blot of Virus Bulk with Specific Antibodies

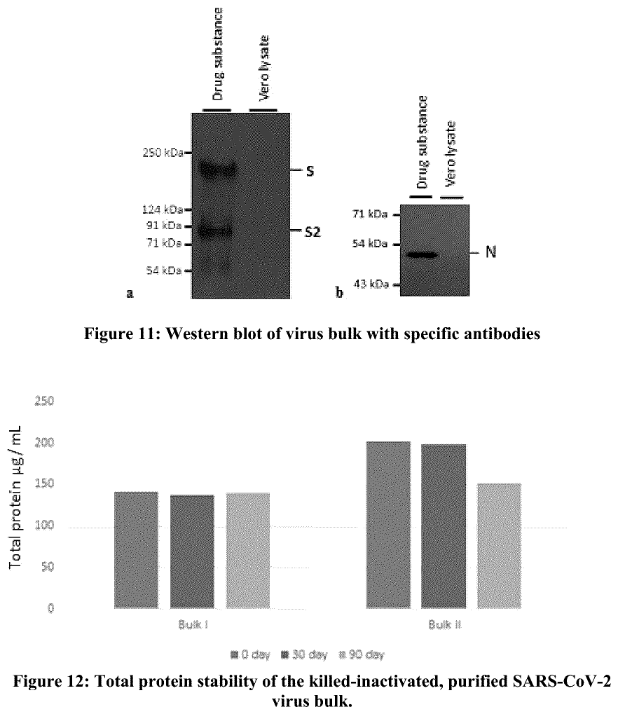

The inactivated and purified SARS-CoV2 (virus bulk or drug substance) and Vero cell lysate were resolved on 10% SDS-Polyacrylamide gels. Western blot with Rabbit polyclonal spike S2-specific antibody (a) and nucleoprotein-specific antibody (b) was carried out. Full length Spike (S) and Spike S2 fragment (S2) were detected by the spike S2-specific antibody while the nucleoprotein (N) was detected by the nucleoprotein-specific antibody. The result is depicted and shown in FIG. 11.

### Example-5: Stability Data

5.1: Total Protein Stability of the Killed-Inactivated, Purified SARS-CoV-2 Virus Bulk:

The protein content in two bulk of killed-inactivated, purified SARS-CoV-2 vaccine were estimated on 0, 30 and 90 days and plotted as bar diagram. The result is depicted and shown in FIG. 12.

5.2: pH Stability of the Killed-Inactivated, Purified SARS-CoV-2 Virus Bulk:

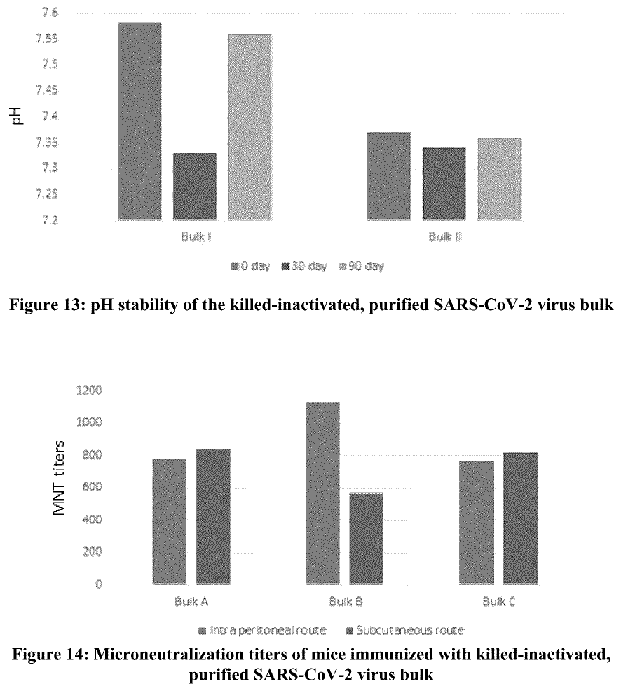

The pH of two bulk of killed-inactivated, purified SARS-CoV-2 vaccine were estimated on 0, 30 and 90 days and plotted as bar diagram. The result is depicted and shown in FIG. 13.

5.1: Microneutralization Titers of Mice Immunized with Killed-Inactivated, Purified SARS-CoV-2 Virus Bulk:

Mice were immunized with 0.5 mL of 20 times diluted bulk samples in intraperitoneal or subcutaneous routes on 0 and 14 days. The SARS-CoV-2 MNT titers of the serum samples after 21 days of immunization were plotted as bar diagram. The result is depicted and shown in FIG. 14.

1. A method for preparation of coronavirus vaccine comprising an inactivated, purified SARS-CoV2 as active ingredient, wherein inactivated purified SARS-CoV2 bulk used in the vaccine composition is prepared by a method comprising the following steps:
(a) using Vero cell line (with or without serum) as cell substrate for SARS-CoV-2 virus culture,
(b) scaling up the SARS-CoV-2 virus culture of step (a) upto a harvest volume of 10L and above,
(c) inactivating the SARS-CoV-2 virus culture of step (b), and
(d) purifying the SARS-CoV-2 virus culture obtained in step (c) and obtaining the inactivated purified SARS-CoV2 bulk.

2. The method for preparation of coronavirus vaccine as claimed in claim 1, wherein the inactivated purified SARS-CoV-2 obtained is killed-inactivated purified SARS-CoV-2.

3. The method for preparation of coronavirus vaccine as claimed in claim 1, wherein the Vero cell line used in step (a) is African Green Monkey Kidney cells.

4. The method for preparation of coronavirus vaccine as claimed in claim 1, wherein the temperature condition in step (a) ranges from 33° C. to 37° C. for the propagation of SARS-CoV2 in mammalian cells.

5. The method for preparation of coronavirus vaccine as claimed in claim 1, wherein the multiplicity of infection of SARS-CoV2 in step (a) ranges from 0.001 to 0.1 for the infection of the mammalian cells.

6. The method for preparation of coronavirus vaccine as claimed in claim 1, wherein a harvest time in step (a) ranges between 48 hours to 80 hours post SARS-CoV2 infection of mammalian cells.

7. The method for preparation of coronavirus vaccine as claimed in claim 1, wherein the inactivation method in step (c) is selected from a group consisting of formalin inactivation, beta propiolactone inactivation, heat inactivation, UV inactivation, gamma radiation inactivation, in the presence or absence of virus stabilizing agents.

8. The method for preparation of coronavirus vaccine as claimed in claim 7, wherein the SARS-CoV-2 virus is inactivated by one of the following methods selected from:
i) Formalin treatment at any concentration ranging from 1:500 upto 1: 4000 v/v of formalin: virus, at 8° C. to 37° C., preferably 25±3° C., for at least 1 to 7 days;
ii) Formalin treatment at any concentration ranging from 1:500 upto 1: 4000 v/v of formalin: virus, at 2° C. to 8° C. for at least 10 to 30 days;
iii) Beta propiolactone at any concentration ranging from 1:500 upto 1: 4000 v/v of BPL: virus, for at least 24 to 48 hrs at temperatures ranging from 8° C. to 30° C., preferably 25±3° C., for 48 hrs;
iv) Beta propiolactone at any concentration ranging from 1:500 upto 1: 4000 v/v of BPL: virus, at 2° C. to 8° C. for at least 3-7 days;
v) A combination of BPL and formalin at any aforementioned conditions, preferably BPL inactivation at 1: 3000 (PBL: virus v/v) for 24 hrs followed by formalin inactivation at 1:3000 (formalin:virus, v/v) for 24 to 48 hrs at 15° C. to 30° C., preferably 25±3° C.

9. The method for preparation of coronavirus vaccine as claimed in claim 8, wherein the SARS-CoV-2 is inactivated by beta-propiolactone ranges between 1:2000 to 1:4000 at 2-8° C. for 24-32 hours.

10. The method for preparation of coronavirus vaccine as claimed in claim 9, wherein the SARS-CoV-2 is inactivated by beta-propiolactone at 1:2500 or 1:4000 ratio at 5+3° C. for 32 hours.

11. The method for preparation of coronavirus vaccine as claimed in claim 1, wherein the purification method in step (d) comprises the use of size exclusion or affinity chromatography resins.

12. The method for preparation of coronavirus vaccine as claimed in claim 11, wherein the size-exclusion chromatography resin comprises Captocore 700 or any other size-exclusion resins for the purification of the SARS-CoV2.

13. The method for preparation of coronavirus vaccine as claimed in claim 11, wherein the affinity chromatography resin comprises Cellufine sulfate or any other affinity resins for the purification of the SARS-CoV2.

14. The method for preparation of coronavirus vaccine as claimed in claim 1, wherein the purification method in step (d) comprises the use of ultracentrifugation.

15. A method of production of inactivated purified SARS-CoV-2 bulk comprising inactivation followed by a combination of size-exclusion or affinity chromatography and tangential flow filtration (TFF).

16. The method as claimed in claim 1, wherein the inactivated purified SARS-CoV-2 obtained in the virus bulk is a killed-inactivated purified SARS-CoV-2 which is used as an active ingredient in the preparation of an immunogenic composition.

17. The method as claimed in claim 16, wherein the immunogenic composition comprises said killed-inactivated purified SARS-CoV-2 as active ingredient, wherein the said composition is formulated as a coronavirus vaccine with or without use of one or more pharmaceutically acceptable excipient(s) as adjuvant and/or stabilizing agents.

18. The method as claimed in claim 17, wherein the adjuvant when used in the vaccine is aluminum hydroxide present in a concentration range of 0.1 mg to 1.5 mg of aluminum per vaccine dose, preferably 0.25 mg to 0.5 mg aluminum per vaccine dose.

19. The method as claimed in claim 17, wherein the stabilizing agents when used are selected from the group consisting of sorbitol, L-glycine, mannitol, L-glutamic acid, human serum albumin and combination thereof.

20. The method as claimed in claim 17, wherein the said immunogenic composition comprises preservatives.

21. The method as claimed in claim 20, wherein the preservative is 2-phenoxyethanol at a concentration of 2.5 to 5 mg/mL.

22. The method as claimed in claim 17, wherein the said immunogenic composition comprises killed-inactivated purified SARS-CoV-2 in buffer comprising of 100 mM Phosphate buffered saline as antigen.

23. The method as claimed in claim 17, wherein the antigen content and pH are stable at −70° C.

24. A coronavirus vaccine obtained by the method as claimed in claim 1.

25. A method for preparation of killed-inactivated purified SARS-CoV2 bulk comprising the following steps:
(a) using Vero cell line (with or without serum) as cell substrate for SARS-CoV-2 virus culture,
(b) scaling up the SARS-CoV-2 virus culture of step (a) upto a harvest volume of 10L and above,
(c) inactivating the SARS-CoV-2 virus culture of step (b), and
(d) purifying the SARS-CoV-2 virus culture obtained in step (c) and obtaining the killed-inactivated purified SARS-CoV2 bulk.

26. A method of inactivation of SARS-CoV-2 virus selected from:
i) Formalin treatment at any concentration ranging from 1:500 upto 1: 4000 v/v of formalin: virus, at 8° C. to 37° C., preferably 25±3° C., for at least 1 to 7 days;
ii) Formalin treatment at any concentration ranging from 1:500 upto 1: 4000 v/v of formalin: virus, at 2° C. to 8° C. for at least 10 to 30 days;
iii) Beta propiolactone at any concentration ranging from 1:500 upto 1: 4000 v/v of BPL: virus, for at least 24 to 48 hrs at temperatures ranging from 8° C. to 30° C., preferably 25±3° C., for 48 hrs;
iv) Beta propiolactone at any concentration ranging from 1:500 upto 1: 4000 v/v of BPL: virus, at 2° C. to 8° C. for at least 3-7 days;
v) A combination of BPL and formalin at any aforementioned conditions, preferably BPL inactivation at 1: 3000 (PBL: virus v/v) for 24 hrs followed by formalin inactivation at 1:3000 (formalin:virus, v/v) for 24 to 48 hrs at 15° C. to 30° C., preferably 25±3° C.

27. A method of treatment and/or prophylaxis for COVID-19 and/or eliciting an immune response against SARS-CoV-2 and its variants in mammals, wherein the said method comprises administration of immunogenic composition comprising killed-inactivated purified SARS-CoV-2 bulk by intranasal, intraperitoneal, oral, intramuscular, subcutaneous or intradermal routes.
